(19) United States
(12) Patent Application Publication
(10) Pub. No.: US 2010/0145367 A1
(43) Pub. Date: Jun. 10, 2010

(54) SYNTHETIC STRUCTURE FOR SOFT TISSUE REPAIR

(76) Inventor: Anthony Ratcliffe, Del Mar, CA (US)

Correspondence Address: CARTER, DELUCA, FARRELL & SCHMIDT, LLP, 445 BROAD HOLLOW ROAD, SUITE 420, MELVILLE, NY, 11747, US

(21) Appl. No.: 12/525,460

(22) PCT Filed: Feb. 13, 2008
(86) PCT No.: PCT/US08/02002

§ 371 Date: Feb. 16, 2010

Related U.S. Application Data
(60) Provisional application No. 60/901,221, filed on Feb. 14, 2007.

(51) Int. Cl. A61B 17/00

(52) U.S. Cl. 606/151

(57) ABSTRACT

Synthetic structures for fibrous soft tissue repair include a planar fibrillar structure which exhibits mechanical properties comparable to those of human fibrous soft tissue. In embodiments, the fibrillar structure possesses at least one secured folded edge portion.

### CROSS-REFERENCE TO RELATED APPLICATIONS

This application claims the benefit of and priority to U.S. Provisional Application Ser. No. 60/901,221, filed Feb. 14, 2007, the entire disclosure of which is incorporated by reference herein.

### BACKGROUND

1. Technical Field

Synthetic structures for the repair of soft tissue are described. Such structures may include, in embodiments, fibrous structures that may be utilized to approximate the physical characteristics of soft tissue and thus may be useful as implants to promote the repair of soft tissue.

2. Background

There are currently several ways in which various types of soft tissues such as ligaments or tendons, for example, are reinforced and/or reconstructed. Suturing the torn or ruptured ends of the tissue is one method of attempting to restore function to the injured tissue. Sutures may also be reinforced through the use of synthetic non-bioabsorbable or bioabsorbable materials. Autografting, where tissue is taken from another site on the patient's body, is another means of soft tissue reconstruction. Yet another means of repair or reconstruction can be achieved through allografting, where tissue from a donor of the same species is used. Still another means of repair or reconstruction of soft tissue is through xenografting, in which tissue from a donor of a different species is used. In addition, bioprosthetic devices for soft tissue attachment, reinforcement, and/or reconstruction have included small intestinal submucosa (SIS) or other naturally occurring extracellular matrix (ECM), and a naturally occurring ECM or ECM component having a synthetic portion coupled thereto.

Using mesh in surgical procedures is well known. For example, surgical mesh may be used to support and/or reinforce a damaged or weakened portion of the body, for example in hernia repair. In this regard, it may be desirable for the mesh to be sufficiently porous to allow for growth of tissue through the graft after implantation. The healing tissue grows through porous openings in the implanted mesh, thereby assimilating the mesh and adding structural integrity to the tissue. Surgical meshes may be produced by knitting, weaving, braiding, or otherwise forming a plurality of yarns into a support trellis. Moreover, such meshes may be produced with monofilament or multifilament yarns made of materials such as polypropylene and polyester. Surgical mesh formed of monofilament yarn provides satisfactory reinforcement ability, but is often stiff and has limited pliability. In contrast, surgical mesh formed of multifilament yarn is often soft and pliable in comparison to mesh formed of monofilament yarn.

### SUMMARY

The present disclosure provides implants which include a planar fibrillar structure having at least one secured folded edge portion. Such an implant may be utilized for human soft tissue repair. The fibrillar structure may exhibit tensile properties of human fibrous soft tissue. In certain embodiments, the fibrillar structure exhibits mechanical properties of human tendons and/or ligaments.

In embodiments, the fibrillar structure exhibits a stiffness of from about 20 to about 80 Newtons per millimeter (N/mm), and exhibits a failure strain at from about 105% to about 150% of its original length.

The fibrillar structure can be woven, can have from about 5 to about 80 warp fibers per inch, and may possess 1 or more layers. The fibrillar structure can include one or more fibers having a diameter of from about 10 microns to about 200 microns. The fibrillar structure can be bioabsorbable or non-bioabsorbable.

Methods of repairing or reconstructing fibrous soft tissue with an implant of the present disclosure are also contemplated. In embodiments, the present disclosure provides methods of providing functional support for a human tendon which include providing an implant including a planar fibrillar structure having at least one secured folded edge portion, wherein the fibrillar structure exhibits mechanical properties of a human tendon, and affixing the fibrillar structure to the human tendon or fragments thereof. In other embodiments, the present disclosure provides methods for replacing the function of a human tendon by providing an implant including a planar fibrillar structure having at least one secured folded edge portion, wherein the fibrillar structure exhibits mechanical properties of a human tendon, and affixing the fibrillar structure to muscles, bones, ligaments, tendons, and fragments thereof.

In some embodiments, methods for providing functional support for a human tendon may include providing an implant including a planar fibrillar structure having at least one secured folded edge portion, wherein the fibrillar structure exhibits mechanical properties of a human tendon, combining the fibrillar structure with a material such as small intestine submucosa biologic graft materials, acellular dermal tissue matrices, and cross-linked pericardium xenografts that have been subjected to an anticalcification process, and affixing the combination to a human tendon or fragments thereof.

Methods for providing functional support for a human ligament are also disclosed which include providing an implant including a planar fibrillar structure having at least one secured folded edge portion, wherein the fibrillar structure exhibits mechanical properties of a human ligament, and affixing the fibrillar structure to the human ligament or fragments thereof. In yet other embodiments, the present disclosure provides methods for replacing the function of a human ligament by providing an implant including a planar fibrillar structure having at least one secured folded edge portion, wherein the fibrillar structure exhibits mechanical properties of a human ligament, and affixing the fibrillar structure to muscles, bones, ligaments, tendons, and fragments thereof.

In some embodiments, methods for providing functional support for a human ligament may include providing an implant including a planar fibrillar structure having at least one secured folded edge portion, wherein the fibrillar structure exhibits mechanical properties of a human ligament, combining the fibrillar structure with a material such as small intestine submucosa biologic graft materials, acellular dermal tissue matrices, and cross-linked pericardium xenografts that have been subjected to an anticalcification process, and affixing the combination to a human ligament or fragments thereof.

### BRIEF DESCRIPTION OF DRAWINGS

FIG. 1 is a depiction of an implant of the present disclosure possessing secured folded edge portions at two opposite ends thereof;

FIG. 2 shows a theoretical strain-stress curve for a biological tissue;

FIG. 3 shows Strain-stress curves for RESTORE® SIS, GRAFTJACKET®, canine infraspinatus (IFS) tendon, and an implant in accordance with the present disclosure;

FIG. 4 shows strain-stress curves for an implant of the present disclosure possessing secured folded edge portions, canine IFS, human IFS, RESTORE®, GRAFTJACKET®, CUFFPATCHT™, and TISSUEMEND®;

FIG. 5 shows the orientation of PLA woven meshes during stress-strain measurements;

FIG. 6 shows strain-stress curves for a variety of materials tested, including RESTORE® SIS, GRAFTJACKET®, a thick mesh, VICRYL®, IFS tendon and various implants in accordance with the present disclosure;

FIG. 7 shows strain-stress curves for implants made with 36 warp and (A) 36 fill, (B) 52 fill, (C) 60 fill fibers demonstrating that the density of fill fibers does not have a significant impact on the tensile stiffness of the mesh; and

FIG. 8 shows strain-stress curves for implants/meshes of the present disclosure with a fill density of 52 fibers/inch with (A) 36 warp, (B) 52 warp, and (C) 60 warp fibers per inch, demonstrating increased tensile properties with increased warp fiber density.

### DETAILED DESCRIPTION OF PREFERRED EMBODIMENTS

A synthetic structure for human fibrous soft tissue repair includes a polymeric fibrous structure that exhibits mechanical properties similar to those possessed by human fibrous soft tissue. In embodiments, the fibrillar structure may be a planar structure which exhibits mechanical properties similar to those possessed by human tendons and/or ligaments. In some embodiments, the planar fibrillar structure exhibits mechanical properties of a human ligament. The mechanical properties of soft tissue and/or the polymeric fibrous structures in accordance with the present disclosure can be determined by any technique within the purview of those skilled in the art. For example, mechanical properties of soft tissue and/or the fibrous structures can be determined by placing a sample in a spring loaded clamp attached to the mechanical testing device and subjecting the sample to constant rate extension (5 mm/min) while measuring load and displacement and recording the resulting strain-stress curve. In embodiments, the polymeric fibrillar structure may exhibit a stiffness comparable to the stiffness exhibited by fibrous soft tissue. In embodiments, a suitable stiffness may be from about 10 to about 500 Newtons per millimeter (N/mm), and suitable tensile strength may be from about 20 to about 2000 Newtons. In some embodiments, the stiffness of the polymeric fibrous structure will be from about 20 to about 80 N/mm. In some embodiments, the fibrillar structure may exhibit a failure strain at from about 105% to about 150% of its original length.

The fibrous structure can be prepared using any method within the purview of those skilled in the art. For example, the fibrous structure can be woven. It is also contemplated that the fibrous structure could be a non-woven structure, provided that it possesses suitable mechanical properties, in embodiments the stiffness, tensile strength, and/or failure strain described above. In certain embodiments, the fibrous structure may be woven and include from about 10 to about 150 warp fibers per inch, in embodiments from about 30 to about 100 warp fibers per inch, in other embodiments, from about 50 to about 75 warp fibers per inch.

The fibrillar structure can be prepared from fibers having a diameter of from about 10 microns to about 1.0 mm, in embodiments from about 15 microns to about 200 microns, in other embodiments from about 20 microns to about 50 microns. The fibrillar structure may be prepared from monofilaments, traditional multifilament yarns, or bi-component multifilament yarns. It is further contemplated that the fibrillar structure can be prepared from multiple fibers of at least two different diameters.

The polymeric fibrillar structure can be made from any biocompatible polymeric material capable of providing suitable mechanical properties. The biocompatible material can be bioabsorbable or non-bioabsorbable. Suitable absorbable materials include, but are not limited to, glycolide, lactide, trimethylene carbonate, dioxanone, caprolactone, alkylene oxides, ortho esters, polymers and copolymers thereof, collagen, hyaluronic acids, alginates, and combinations thereof. Suitable non-absorbable materials include, but are not limited to, polypropylene, polyethylene, polyamide, polyalkylene therephalate (such as polyethylene therephalate, polybutylene therephalate, and the like), polyvinylidene fluoride, polytetrafluoroethylene, and blends and copolymers thereof.

The dimensions of the fibrillar structure are not critical. The width and length dimensions of the fibrous structure can vary within those ranges conventionally used for a specific application and delivery device. For example, such ranges include dimensions of about 1 centimeter by about 1 centimeter, to about 15 centimeters by about 15 centimeters. In some embodiments, a thin mesh may be formed having a thickness from about 0.05 millimeters to about 1.0 millimeters, in embodiments from about 0.1 millimeters to about 0.75 millimeters. The present fibrillar structures can advantageously be dimensioned to allow them to be rolled or otherwise folded so as to fit within a cannula having a small diameter to allow arthroscopic or laparoscopic implantation. In embodiments, the fibrillar structures in accordance with the present disclosure may define openings on the order of from about 0.5 mm to about 2 mm, in embodiments from about 0.7 mm to about 1.3 mm.

In embodiments, a fibrillar structure of the present disclosure may have at least one secured, folded edge portion, i.e., an edge of the fibrillar structure of the present disclosure may be folded over and affixed to the body of the fibrillar structure. In embodiments, this secured, folded edge portion may be referred to as a “hem.” The secured, folded edge portion of the fibrillar structure may be formed by folding over a free edge of the fibrillar structure and affixing the free edge to the rest of the fibrillar structure, i.e., the body of the fibrillar structure, thereby resulting in the fibrillar structure possessing an edge hemmed to the rest of the body of the fibrillar structure. The folded edge portion of the fibrillar structure thus produces an end of the fibrillar structure that has two layers, i.e., the fibrillar structure folded over onto itself, for a distance to the edge of the fibrillar structure. In some embodiments, opposite edges of a fibrillar structure of the present disclosure may both be folded over and affixed to the body of the fibrillar structure so that two opposite ends of the fibrillar structure possess secured, folded edge portions.

The free edges of the fibrillar structure of the present disclosure may be attached to the body of said structure utilizing methods within the purview of those skilled in the art including, but are not limited to, the use of stitches, ultrasonic welding, heat, adhesives, combinations thereof, and the like. The placement of these securement means may be along any part of the folded edge portion of the fibrillar structure.

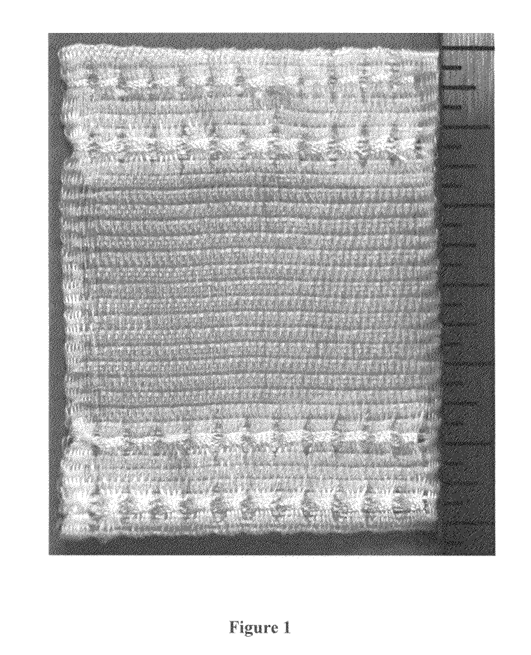

In some embodiments, the folded edge may be attached to the body of the fibrillar structure by utilizing stitches. Such stitches may include a thread or filament made of any biocompatible material within the purview of those skilled in the art, including those materials identified above as suitable for forming the fibrillar structure of the present disclosure. In embodiments, multiple rows of stitches may be utilized to form the hem on an edge of the fibrillar structure of the present disclosure. Such a structure is depicted, for example, in FIG. 1, which shows a fibrillar structure 10 of the present disclosure having two opposite edges folded over forming opposite ends 20 and 30 of fibrillar structure 10. As depicted in FIG. 1, two rows of stitches 24 and 26 are present at end 20 of the fibrillar structure 10 with an additional two rows of stitches 34 and 36 present at end 30 of the fibrillar structure 10. In other embodiments, not shown, a single row of stitches may be utilized to secure the folded edge of fibrillar structure 10, e.g., stitches 24 or 26 along may be placed along end 20, optionally in combination with stitches 34 or 36 along end 30.

As noted above, in other embodiments heat may be utilized to affix a free edge of the fibrillar structure to the body of the fibrillar structure. The amount of heat applied should be sufficient to permit at least the free edge of the fibrillar structure to become tacky so that it may be affixed to the body of the fibrillar structure. In some embodiments, the amount of heat may render both the edges and the body of the fibrillar structure tacky thereby permitting the free edge to be affixed to the body of the fibrillar structure. Suitable temperatures which may be utilized to affix the free edge to the body of the fibrillar structure are within the purview of those skilled in the art and may vary depending upon the material utilized to form the fibrillar structure. In embodiments, a suitable temperature may be from about 50° C. to about 500° C., in embodiments from about 100° C. to about 400° C.

In other embodiments, adhesives may be used to affix a free edge of the fibrillar structure to the body of the fibrillar structure. Suitable adhesives may be, in embodiments, any biocompatible material within the purview of those skilled in the art. Examples of such adhesives include, but are not limited to, adhesives containing fibrin, bioabsorbable gelatins, acrylics, acrylates, natural rubbers, polysaccharides, peptides, polypeptides, polyalkylene glycols, substituted variations thereof, combinations thereof, and the like.

In use, the fibrillar structure may be attached to tissue utilizing any method within the purview of those skilled in the art, including the use of sutures, tacks, adhesives, combinations thereof, and the like. Turning to FIG. 1, in embodiments end 20 of fibrillar structure 10 may be affixed to tissue by placing a line of sutures between rows of stitches 24 and 26 thereby attaching end 20 of fibrillar structure 10 to tissue; similarly, end 30 of fibrillar structure 10 may be affixed to tissue by placing a line of sutures between rows of stitches 34 and 36 thereby attaching end 30 of fibrillar structure 10 to tissue.

In embodiments, the formation of a secured, folded edge portion of the fibrillar structure as described above may provide a fibrillar structure of the present disclosure with enhanced strength at the point of attachment and may minimize the chance that the fibrillar structure of the present disclosure may become detached from the sutures or similar means utilized to affix a fibrillar structure of the present disclosure to tissue.

It is further contemplated that a bioactive agent can be applied to the fibrillar structure. The term “bioactive agent”, as used herein, is used in its broadest sense and includes any substance or mixture of substances that have clinical use. Bioactive agents may or may not have pharmacological activity, e.g., as a dye, or fragrance. Alternatively, bioactive agents may provide a therapeutic or prophylactic effect. For example, bioactive agents may affect or participate in tissue growth, cell growth, cell differentiation, and the like, and may also be able to invoke a biological action such as an immune response or play any other role in one or more biological processes.

Examples of classes of bioactive agents which may be utilized in accordance with the present disclosure include anti-adhesives, antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, and enzymes. It is also intended that combinations of bioactive agents may be used.

Anti-adhesive agents can be used to prevent adhesions from forming between the fibrillar structures of the present disclosure and the surrounding tissues opposite the target tissue. In addition, anti-adhesive agents may be used to prevent adhesions from forming between the fibrillar structures of the present disclosure and any packaging material. Some examples of these agents include, but are not limited to poly(vinyl pyrrolidone), carboxymethyl cellulose, hyaluronic acid, polyethylene oxide, poly vinyl alcohols and combinations thereof.

Suitable antimicrobial agents which may be included as a bioactive agent with a fibrillar structure of the present disclosure include triclosan, also known as 2,4,4′-trichloro-2′-hydroxydiphenyl ether, chlorhexidine and its salts, including chlorhexidine acetate, chlorhexidine gluconate, chlorhexidine hydrochloride, and chlorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bacitracin, neomycin, chloramphenicol, miconazole, quinolones such as oxolinic acid, norfloxacin, nalidixic acid, pefloxacin, enoxacin and ciprofloxacin, penicillins such as oxacillin and pipracil, nonoxynol 9, fusidic acid, cephalosporins, and combinations thereof. In addition, antimicrobial proteins and peptides such as bovine lactoferrin and lactoferricin B may be included as a bioactive agent with a fibrillar structure of the present disclosure.

Other bioactive agents which may be included as a bioactive agent with a fibrillar structure of the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g. oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; estrogens; antibacterials; antibiotics; anti-fungals; anti-virals; anticoagulants; anticonvulsants; antidepressants; antihistamines; and immunological agents.

Other examples of suitable bioactive agents which may be included with a fibrillar structure of the present disclosure include viruses and cells, peptides, polypeptides and proteins, analogs, muteins, and active fragments thereof, such as immunoglobulins, antibodies, cytokines (e.g. lymphokines, monokines, chemokines), blood clotting factors, hemopoietic factors, interleukins (IL-2, IL-3, IL-4, IL-6), interferons (β-IFN, (α-IFN and γ-IFN), erythropoietin, nucleases, tumor necrosis factor, colony stimulating factors (e.g., GCSF, GM-CSF, MCSF), insulin, anti-tumor agents and tumor suppressors, blood proteins, gonadotropins (e.g., FSH, LH, CG, etc.), hormones and hormone analogs (e.g., growth hormone), vaccines (e.g., tumoral, bacterial and viral antigens); somatostatin; antigens; blood coagulation factors; extracellular matrix molecules such as fibronectin and laminin; hyaluronic acid; collagens; glycosaminoglycans; morphogens; chemoattractants; growth factors (e.g., nerve growth factor, insulin-like growth factor, EGF, FGF, PDGF and VEGF); protein inhibitors, protein antagonists, and protein agonists; nucleic acids, such as antisense molecules, DNA and RNA; oligonucleotides; polynucleotides; and ribozymes.

The bioactive materials can be applied to the fibrillar structure using any technique within the purview of those skilled in the art. For example, the bioactive agent may be applied to the fibrillar structure of the present disclosure in any suitable form of matter, e.g., films, powders, liquids, gels and the like. In embodiments, a solution of the bioactive agent in a suitable solvent can be prepared and the solvent driven off to leave the bioactive material deposited on the fibrillar structure. A further example is a bioactive agent that can be crosslinked around the fibrillar structure so as to embed the fibrillar structure within the bioactive agent.

Where a secured, folded edge portion of the fibrillar structure is formed by folding over an edge of the fibrillar structure and attaching same to the body of the fibrillar structure, a bioactive material may also be placed within the folded edge portion of the fibrillar structure. The bioactive agent may be placed anywhere within the folded edge portion of the fibrillar structure, in embodiments in the channel or crease formed where the edge of the fibrillar structure is folded over. Any bioactive material described above may be placed within the folded edge of the fibrillar structure. In this manner, bioactive agents may be released at the site of attachment of the fibrillar structure, in embodiments the defect itself being treated, thereby enhancing healing of the defect.

In embodiments, the bioactive material may be placed in a tube structure which, in turn, is placed within the folded edge portion of the fibrillar structure, including any channel or crease formed where the edge of the fibrillar structure is folded. Any biocompatible material within the purview of those skilled in the art may be utilized to form a tube within which a bioactive material may be placed. Such materials include, in embodiments, any material utilized to form the fibrillar structure of the present disclosure.

It is further contemplated that more than one layer of fibrillar structure in accordance with the present disclosure can be combined to prepare a soft tissue repair device in accordance with other embodiments. Each of the two or more layers may have the same or different mechanical properties, provided that the combination of the two or more layers exhibit mechanical properties of soft tissue. In addition, each of the two or more layers may have the same or different bioabsorbability properties. In addition, each of the two or more layers may optionally have the same or different bioactive materials applied thereto. As noted above, where the fibrillar structure is of a single layer, the folded edge portion of the fibrillar structure produces an end of the fibrillar structure that has two layers; similarly, where the body of the fibrillar structure possesses multiple layers, the secured, folded edge portion will have twice the number of layers because of the folding of the fibrillar structure onto itself, for a distance to the edge of the fibrillar structure.

The fibrillar structure can be packaged and sterilized in accordance with any of the techniques within the purview of those skilled in the art. The package in which the implant or plurality of implants are maintained can take a variety of forms within the purview of those skilled in the art. The packaging material itself can be bacteria and fluid or vapor impermeable, such as a film, sheet, or tube made of polyethylene, polypropylene, poly(vinylchloride), poly(ethylene terephthalate), and the like. Scams, joints, seals, and the like may be formed in such packaging by conventional techniques, such as, for example, heat sealing and adhesive bonding. Examples of heat sealing include sealing through the use of heated rollers, sealing through use of heated bars, radio frequency sealing, and ultrasonic sealing. Peelable seals based on pressure sensitive adhesives may also be used.

The fibrillar structures described herein can be used to repair, support, and/or reconstruct fibrous soft issue. The fibrillar structures may rapidly restore mechanical functionality to the fibrous soft tissue. The fibrillar structures may be implanted using conventional surgical or laparoscopic/arthroscopic techniques. The fibrillar structure can be affixed to the soft tissue or to bone adjacent to or associated with the soft tissue to be repaired. In embodiments, the fibrillar structure may be affixed to muscle, bone, ligament, tendon, or fragments thereof. Affixing the fibrillar structure can be achieved using techniques within the purview of those skilled in the art using, for example, sutures, staples, and the like, with or without the use of appropriate anchors, pledgets, etc.

The present fibrillar structure can be used alone or in combination with other tissue repair products within the purview of those skilled in the art. Suitable tissue repair products that can be used in combination with the present fibrillar structures include, for example, RESTORE® a small intestine submucosa (SIS) biologic graft material that is commercially available from Depuy Orthopedics Inc., Warsaw IN; GRAFTJACKE®, an acellular dermal tissue matrix commercially available from Wright Medical Technology, Inc., Arlington, Tenn.; CUFFPATCHT™ Type I porcine collagen material from Biomet Sports Medicine, Inc./Arthrotek (Warsaw, Ind.); TISSUEMEND® acellular collagen membrane materials from Stryker (Kalamazoo, Mich.); and ENCUFF® a cross-linked pericardium xenograft that has been subjected to an anticalcification process commercially available from Selhigh, Inc., Union NJ. Other tissue repair products suitable for use in connection with the present fibrillar structures will be apparent to those skilled in the art. The other tissue repair product can be separate from or attached to the fibrillar structure.

In order that those skilled in the art may be better able to practice the compositions and methods described herein, the following examples are given as an illustration of the preparation of the present compositions and methods. It should be noted that the invention is not limited to the specific details embodied in the examples.

### Example 1

### Constant Rate Extension Test of Polylactic Acid Thin Woven Mesh

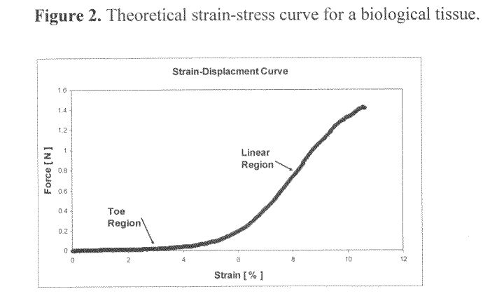

The purpose of this experiment was to determine the mechanical properties of a thin woven polylactic acid (PLA) consisting of 52 Warp by 52 Fill fibers compared to RESTORE® a small intestine submucosa (SIS) biologic graft material that is commercially available from Depuy Orthopedics Inc., Warsaw IN, GRAFTJACKET® an acellular dermal tissue matrix commercially available from Wright Medical Technology, Inc., Arlington, Tenn. and canine infraspinatus (IFS) tendon. As illustrated in FIG. 2, as biological tissues are extended there may be two regions over which the mechanical properties may be drastically different; a toe region where the matrix components may be crimped or unorganized; and a linear region where the matrix components may align in the direction of extension leading to increased loads during extension.

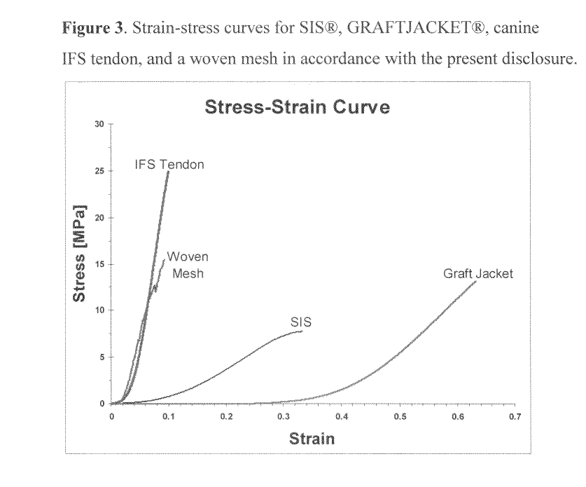

The samples were placed in a spring loaded clamp attached to the mechanical testing device and subjected to constant rate extension (5 mm/min), while measuring load and displacement. The strain-stress curve was recorded for each sample and the data were compared to that obtained for IFS tendon. As seen in FIG. 3, the tensile properties of the thin woven mesh is comparable to that of the IFS tendon.

### Example 2

Additional comparisons were conducted utilizing the methods of Example 1 above between a mesh of the present disclosure possessing a hem as depicted in FIG. 1 and canine IFS tendon, human IFS tendon, RESTORE® SIS graft material, GRAFTJACKET® acellular dermal tissue matrix, CUFFPATCHT™ Type I porcine collagen material from Biomet Sports Medicine, Inc./Arthrotek (Warsaw, Ind.), and TISSUEMEND® acellular collagen membrane materials from Stryker (Kalamazoo, Mich.).

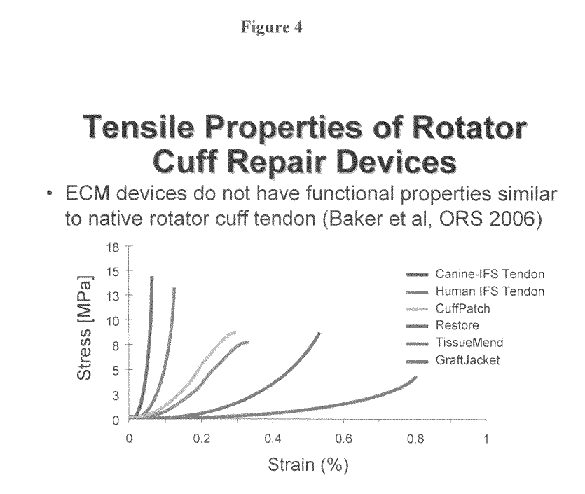

The results of these tests are set forth in FIG. 4. As seen in FIG. 4, the tensile properties of the mesh of the present disclosure possessing a hem were similar to those of the IFS tendons.

### Example 3

### Constant Rate Extension Test of Polylactic Acid Woven Meshes of Varying Warp and Fiber Structures

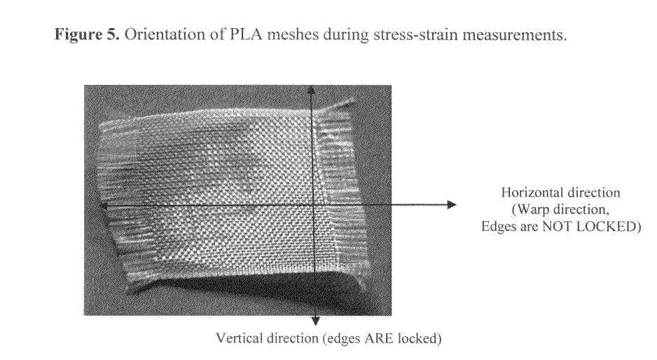

The purpose of this experiment was to determine the mechanical properties of a series of woven polylactic acid (PLA) meshes constructed with a defined number of warp and fill fibers. Included in the study were samples of human and canine infraspinatus (IFS) tendon to provide a comparison of the strength of natural tendon. The data can be used to develop a relationship between the number of warp and fill fibers required to design meshes with mechanical properties approximating human or canine IFS tendon. The samples were tested in a mechanical testing device under identical conditions. All of the meshes were tested in their horizontal direction wherein the ends of the fibers were not locked (Sec FIG. 5).

The samples were placed in a spring loaded clamp attached to the mechanical testing device and subjected to constant rate extension (5 mm/min), while measuring load and displacement. The data were analyzed to determine ramp modulus (stiffness) and strain at a load of 2 Newtons (N). The ramp modulus was computed between 25 and 75% of the maximum load recorded. In addition, the strain-stress curve was recorded for each sample and the data were compared to that obtained for human and canine IFS tendon. Table 1 shows the numbers of warp and fill fibers for the samples tested in this study.

TABLE 1
Different mesh designs involved in the study

| Scaffold's Number, | Number of Warp (W) | Number of Fill (F) |
|---|---|---|
| Number of Samples | Fibers | Fibers |
| 1, n = 1 | 36 | 36 |
| 2, n = 1 | 36 | 52 |
| 3, n = 1 | 36 | 60 |
| 4, n = 1 | 52 | 36 |
| 5, n = 3 | 52 | 52 |
| 6, n = 1 | 52 | 60 |
| 7, n = 1 | 60 | 36 |
| 8, n = 1 | 60 | 52 |
| 9, n = 1 | 60 | 60 |

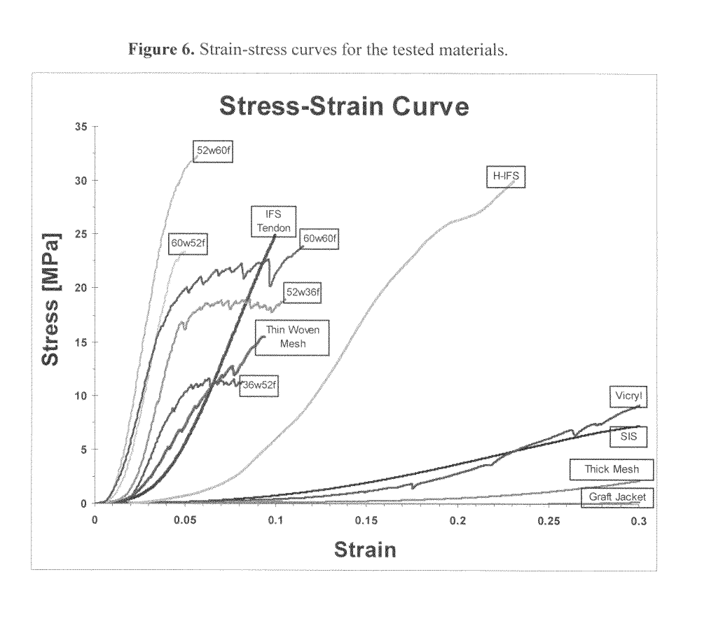

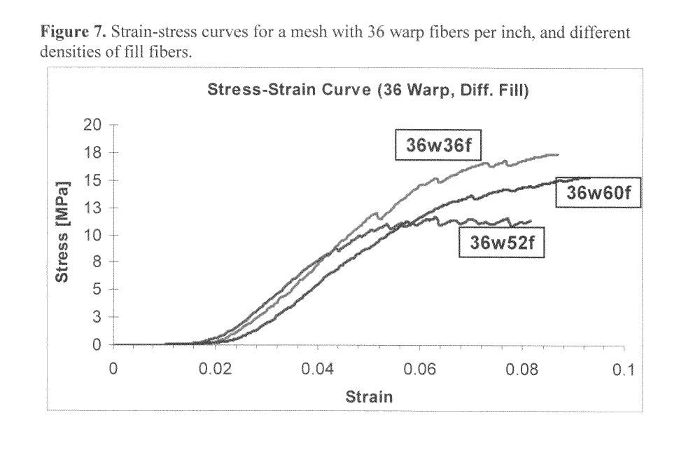

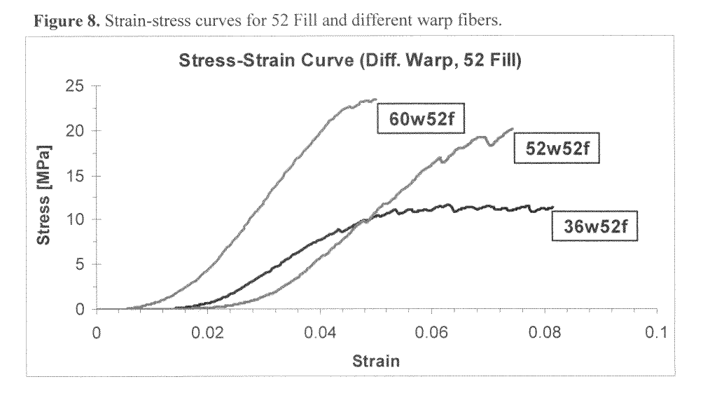

The strain-stress curves for some of the materials tested are shown in FIG. 6. The results demonstrate that the mechanical properties of the meshes can be greater than, similar to, or less than human and canine IFS tendon, depending on the fabrication of the mesh. In FIG. 7 the stain-stress curves are grouped according to the number of warp fibers to examine a potential relationship between the mesh architecture and the resulting mechanical properties. The results show that the number of fibers in the fill direction do not significantly affect the tensile properties of the fibrillar structure. Since the constant rate extension test is done in the warp direction, the fibers in the fill direction should not contribute to the strength of the mesh. As can be seen in FIG. 8, the higher number of warp fibers resulted in a steeper slope in the linear region of the graph which is confirmed with the average ramp modulates of 356, 557, and 562 MPa for 36, 52 and 60 warp fibers respectively.

Since the constant rate extension test is done in the warp direction the increase in the number of warp fibers when there is the same number of fill fibers should result in an increase in the strength of the mesh in that direction.

Table 2 shows the maximum loads for each of the mesh dimensions tested. Those skilled in the art know that the maximum load tolerated by the rotator cuff tendon is in the range of 550-1,800 N. In addition, a synthetic tendon should have a strength with a minimum value of approximately 40% of the lower range or about 220 N, to perform in the functional mechanical range of a rotator cuff tendon. Therefore, according to the data in Table 2, a 2 inch wide repair device would require about 2-3 layers of mesh to satisfy the load requirement. As those skilled in the art will appreciate, the number of layers required depends on the selected warp and fill fiber numbers. In some cases, a slight increase in the width of only one layer, for example from 2 inches to 3.2 inches would satisfy the maximum load tolerated by the mesh.

TABLE 2
Maximum load tolerated with each mesh

| | | | Max | Max Load 1 | Max Load 2 | Max Load 3 |
|---|---|---|---|---|---|---|
| | Width | Max load | load | layer, 2″ wide | layers, 2″ wide | layers, 2″ wide |
| #W × #F | [mm] | [g] | [N] | [N] | [N] | [N] |
| 36 × 36 | 10.16 | 1607.82 | 15.8 | 79 | 158 | 237 |
| 36 × 52 | 11.25 | 675.37 | 6.6 | 30 | 60 | 90 |
| 36 × 60 | 12.45 | 1294.92 | 12.7 | 52 | 104 | 155 |
| 52 × 36 | 9.56 | 1519.71 | 14.9 | 79 | 159 | 238 |
| 52 × 52 (1) | 10.25 | 1552.50 | 15.2 | 75 | 151 | 226 |
| 52 × 52 (2) | 10.18 | 959.24 | 9.4 | 47 | 94 | 141 |
| 52 × 52 (3) | 9.92 | 1177.77 | 11.6 | 59 | 118 | 178 |
| 52 × 60 | 10.00 | 2682.20 | 26.3 | 134 | 267 | 401 |
| 60 × 36 | 11.00 | 1061.37 | 10.4 | 48 | 96 | 144 |
| 60 × 52 | 10.80 | 2145.01 | 21.0 | 99 | 198 | 297 |
| 60 × 60 | 11.38 | 3125.52 | 30.7 | 137 | 274 | 411 |
| C-SSP Tendon | 12.14 | 895.15 | 8.8 | 4.12 | N/A | N/A |
| C-SubS Tendon | 12.21 | 622.30 | 6.1 | 1.41 | N/A | N/A |

The data shown above can be used to calculate the maximum tolerated load for each warp fiber as a function of the number of fill fibers (Table 3). As can be seen from the data in Table 3, the maximum load per warp fiber is very similar for the meshes with different numbers of fill fibers. The average maximum load is 0.764 N and therefore to construct a mesh that would tolerate a maximum load of 220 N would require 228 Warp fibers.

TABLE 3
Maximum load for each Warp fiber

| | Max Load | | Max Load/# Warp |
|---|---|---|---|
| | for Warp × | Max Load/# Warp | AVERAGE |
| Description | Ave. Fill [N/in] | [N/Warp Fiber] | [N/Warp Fiber] |
| 36 W × | 27 | 0.744 | 0.764 |
| Average Fill | | | |
| 52 W × | 39 | 0.759 | |
| Average Fill | | | |
| 60 W × | 47 | 0.789 | |
| Average Fill | | | |

The conclusions from this study include that the meshes can be purposely designed to have specific mechanical properties, and these can be similar to the mechanical properties of human and canine IFS tendon. Therefore, the meshes would be of sufficient strength to repair a human rotator cuff tendon injury. Specifically, it was determined that the number of warp fibers influences the maximum tolerated load and that the load tolerated per warp fiber is approximately 0.764 N. These data provide the information required to select the width of a mesh to affect a desired tendon repair and determine the number of warp fibers required to provide the necessary maximum tolerated load.

It will be understood that various modifications may be made to the embodiments disclosed herein. Therefore, the above description should not be construed as limiting, but merely as exemplifications of preferred embodiments. Those skilled in art will envision other modifications within the scope and spirit of the claims appended hereto.

What is claimed is:

1. An implant comprising: a planar fibrillar structure having at least one secured folded edge portion.

2. The implant of claim 1, wherein the planar fibrillar structure exhibits mechanical properties of human fibrous soft tissue.

3. The implant of claim 1, wherein two opposite edges of the fibrillar structure form secured folded edge portions.

4. The implant of claim 1, wherein the secured folded edge portion is secured to the fibrillar structure by stitches, ultrasonic welding, heat, adhesives, or combinations thereof.

5. The implant of claim 1, wherein the secured folded edge portion of the fibrillar structure is affixed to the fibrillar structure by at least one row of stitches.

6. The implant of claim 1, wherein the secured folded edge portion of the fibrillar structure is affixed to the fibrillar structure by an adhesive selected from the group consisting of fibrins, bioabsorbable gelatins, acrylics, acrylates, natural rubbers, polysaccharides, peptides, polypeptides, polyalkylene glycols, and combinations thereof.

7. The implant of claim 1, wherein the planar fibrillar structure is bioabsorbable.

8. The implant of claim 7, wherein the planar fibrillar structure is fabricated from at least a member selected from the group consisting of glycolide, lactide, trimethylene carbonate, dioxanone, caprolactone, alkylene oxides, ortho esters, collagen, hyaluronic acids, alginates, and combinations thereof.

9. The implant of claim 1, wherein the planar fibrillar structure is non-bioabsorbable.

10. The implant of claim 9 wherein the planar fibrillar structure is fabricated from at least a member of the group consisting of polypropylene, polyethylene, polyamide, polyalkylene therephalate, polyvinylidene fluoride, polytetrafluoroethylene and combinations thereof.

11. The implant of claim 1, wherein the planar fibrillar structure exhibits the mechanical properties of a human tendon.

12. The implant of claim 11, wherein the planar fibrillar structure exhibits a stiffness of from about 10 to about 500 Newtons per millimeter.

13. The implant of claim 11, wherein the planar fibrillar structure exhibits a tensile strength of from about 20 to about 2000 Newtons.

14. The implant of claim 11, wherein the planar fibrillar structure exhibits a failure strain at from about 105% to about 150% of its original length.

15. The implant of claim 1, wherein the planar fibrillar structure exhibits mechanical properties of a human ligament.

16. The implant of claim 15, wherein the planar fibrillar structure exhibits a stiffness of from about 10 to about 500 Newtons per millimeter N/mm.

17. The implant of claim 15, wherein the planar fibrillar structure exhibits a tensile strength of from about 20 to about 2000 Newtons.

18. The implant of claim 15, wherein the polymeric fibrous structure exhibits a failure strain at 105% to about 150% of its original length.

19. The implant of claim 1, wherein the planar fibrillar structure has from about 10 to about 150 warp fibers per inch.

20. The implant of claim 1, wherein the planar fibrillar structure is knitted.

21. The implant of claim 1, wherein the planar fibrillar structure is woven.

22. The implant of claim 1, wherein the planar fibrillar structure is non-woven.

23. The implant of claim 1, wherein the planar fibrillar structure comprises at least one fiber having a diameter from about 10 microns to about 200 microns.

24. The implant of claim 1, wherein the planar fibrillar structure comprises at least two fibers of different diameters.

25. The implant of claim 1, wherein the planar fibrillar structure has at least two layers.

26. The implant of claim 1, wherein the planar fibrillar structure includes a bioactive agent thereon.

27. The implant of claim 1, wherein the planar fibrillar structure includes a bioactive agent within the at least one secured folded edge portion.

28. A method of providing functional support for a human tendon comprising:
providing an implant comprising a planar fibrillar structure having at least one secured folded edge portion, wherein the fibrillar structure exhibits mechanical properties of a human tendon; and
affixing the fibrillar structure to the human tendon or fragments thereof.

29. A method of replacing the function of a human tendon comprising:
providing an implant comprising a planar fibrillar structure having at least one secured folded edge portion, wherein the fibrillar structure exhibits mechanical properties of a human tendon; and
affixing the fibrillar structure to a member of the group selected from the group consisting of muscle, bone, ligament, tendon, and fragments thereof.

30. A method of providing functional support for a human ligament comprising:
providing an implant comprising a planar fibrillar structure having at least one secured folded edge portion, wherein the fibrillar structure exhibits mechanical properties of a human ligament; and
affixing the fibrillar structure to the human ligament or fragments thereof.

31. A method of replacing the function of a human ligament comprising:
providing an implant comprising a planar fibrillar structure having at least one secured folded edge portion, wherein the fibrillar structure exhibits mechanical properties of a human ligament; and
affixing the fibrillar structure to a member of the group selected from the group consisting of muscle, bone, ligament, tendon, and fragments thereof.

32. A method of providing functional support for a human tendon comprising:
providing an implant comprising a planar fibrillar structure having at least one secured folded edge portion, wherein the fibrillar structure exhibits mechanical properties of a human tendon;
combining the fibrillar structure with a member selected from the group consisting of small intestine submucosa biologic graft materials, acellular dermal tissue matrices, and cross-linked pericardium xenografts that have been subjected to an anticalcification process; and
affixing the combination to a human tendon or fragments thereof.

33. A method of providing functional support for a human ligament comprising:
providing an implant comprising a planar fibrillar structure having at least one secured folded edge portion, wherein the fibrillar structure exhibits mechanical properties of a human ligament;
combining the fibrillar structure with a member selected from the group consisting of small intestine submucosa biologic graft materials, acellular dermal tissue matrices, and cross-linked pericardium xenografts that have been subjected to an anticalcification process; and
affixing the combination to a human ligament or fragments thereof.
